(19) United States
(12) Patent Application Publication
(10) Pub. No.: US 2020/0107859 A1
(43) Pub. Date: Apr. 9, 2020

(54) CANNULA COMPRISING MOVABLE & INFLATABLE BALLOON

(71) Applicant: 5RMED TECHNOLOGY (CHENGDU) CO., LTD, Chengdu (CN)

(72) Inventor: Moshu ZHU, Chengdu (CN)

(21) Appl. No.: 16/701,168

(22) Filed: Dec. 3, 2019

Related U.S. Application Data
(63) Continuation of application No. PCT/CN2018/089176, filed on May 31, 2018.

(30) Foreign Application Priority Data
Jun. 3, 2017 (CN) 201710415787.1

(51) Int. Cl. A61B 17/34

(52) U.S. Cl. CPC A61B 17/3498; A61B 17/3423; A61B 2017/3441; A61B 2017/3419; A61B 2017/3486

(57) ABSTRACT

This application discloses a cannula comprising the movable and inflatable balloon, which includes the first seal assembly and the second seal assembly, which comprises the lower outer casing and the hollow sleeve connected thereto and extending to the distal end. The first seal assembly, the second seal assembly and the hollow sleeve include the instrument access that is communicated and substantially aligned, the hollow sleeve including the inner cylinder surface, the outer cylinder surface and the sleeve-wall therebetween; the distal end of the hollow sleeve further includes the open sleeve lip; the cannula further includes the inflatable balloon assembly, the one-way valve assembly for inflation and deflation, and the gas access connecting the balloon assembly and the one-way valve assembly; the balloon assembly includes the balloon lip and the balloon body coupled thereto.

### CROSS-REFERENCE TO RELATED APPLICATIONS

This application is a continuation of International Patent Application No. PCT/CN2018/089176 with a filing date of May 31, 2018, designating the United States, now pending, and further claims priority to Chinese Patent Application No. 201710415787.1 with a filing date of Jun. 3, 2017. The content of the aforementioned applications, including any intervening amendments thereto, are incorporated herein by reference.

### TECHNICAL FIELD

The present application relates to a minimally invasive surgical instrument, and in particular, to a trocar comprising an inflatable balloon.

### BACKGROUND OF THE INVENTION

A trocar is a surgical instrument that is used to establish an artificial access in minimally invasive surgery (especially in rigid endoscopy). A trocar assembly generally comprise in general a cannula and an obturator. The general clinical use is as follows: firstly cut a small incision on the patient's skin, and then pass the obturator through the cannula, the distal end of the obturator exceeds the distal end of the cannula, and then through the skin opening penetrating the body wall into the body cavity. Once penetrated into the body cavity, the obturator is removed, and the cannula will be left as access for the instrument get in/out of the body cavity.

In rigid endoscopy surgery, especially in laparoscopic surgery, the pneumoperitoneum is usually used to continuously infuse the patient's abdominal cavity with gas (such as carbon dioxide) and maintain a stable pressure (about 13-15 mmHg) to obtain the sufficient operation space. The cannula comprises a sleeve, an outer body, a seal membrane (also known as instrument seal) and a duck bill (also known as closure valve). Said sleeve provides an access for the instrument in/out of the body cavity, said outer body connecting the sleeve, the duck bill and the seal membrane into a sealing system; said duck bill normally not providing sealing for the inserted instrument, but automatically closing and forming a seal when the instrument is removed; said seal membrane accomplishing a gas-tight seal against the instrument when it is inserted.

At present, the common most procedures used in laparoscopy to entry into the peritoneal cavity: open technique (Hasson technique) and closed technique (Veress needle). Hasson technique is mainly used for patients with the adhesions in the abdominal wall. Hasson technique usually first makes a 2 cm incision along the upper or lower edge of the umbilicus. The incision crosses through the entire abdominal wall, and then enter the entire through the incision by the finger to separate the adhesion between the abdominal wall and the omentum or the intestinal canal; Hasson cannula was then inserted under direct visualization and carbon dioxide gas was injected into the patient's abdominal cavity through Hasson cannula to form a pneumoperitoneum. The closed technique is also called the direct puncture technique, that is, only the epidermis of the abdominal wall of the patient puncture position is made into a small incision, and then the obturator is passed through the cannula, and punctured the abdominal wall into the body through the small incision.

Hasson cannula disclosed in the prior art is mainly divided into three categories. The first one, for example, the cannula with the hinge structure disclosed in U.S. Pat .No. 5,203,773, which is inflated and fixed by the hinge, is gradually abandoned due to easy leakage. The second one, such as the Hassan cannula, which is composed of a conical shaped collar and a smooth sleeve assembly disclosed in U.S. Pat. No. 5,259,973. First, sutures the conical collar in the incision, and then fixes the smooth cannula to the conical collar. It is widely used because of its low cost, but its operation is relatively complicated and causes secondary injury to the patient. The third one, the cannula with the inflation balloon, for example, disclosed in U.S. Pat. Nos. 5,468, 248, 6, 904, 454, and 8,888, 892. The inflation member can be selectively inflated by the syringe to secure the cannula to the abdominal wall, and the deflation releases and reduces the balloon to facilitate the cannula got in and out through the patient's incision. The inflated balloon firmly secures the cannula to the patient's incision and achieves sealing of the contact region with less damage to the patient's wound. However, such cannula comprising the balloon is complicated in structure, with high cost, and expensive.

The sleeve assembly with the balloon is generally only used in the field of Hasson technique, and the cannula with the balloon that has been disclosed and commercialized so far is basically not used in the direct puncture technique. The resistance to puncturing the patient's body wall by the trocar with the cannula with the balloon is too large, which is not conducive to the surgeon control or has the risk of puncturing the internal organs of the patient. Compared to the cannula without the balloon, the cannula with the balloon is favored by the surgeon because of its more secure attachment to the abdominal wall of the patient. However the puncture force of the cannula with the balloon greatly limits the cannula with the balloon in the field of the direct puncture technique. Moreover, the cannula with the balloon that has been disclosed so far and has been commercialized has not properly solved the problem of the large puncture force.

### SUMMARY

In order to solve one or more technical problems in the prior art, a cannula comprising the movable and inflatable balloon, provided in the present invention, includes the first seal assembly and the second seal assembly, which comprises the lower outer casing and the hollow sleeve connected thereto and extending to the distal end. The first seal assembly, the second seal assembly and the hollow sleeve include an instrument access that is communicated and substantially aligned, the hollow sleeve including an inner cylinder surface, an outer cylinder surface and a sleeve-wall therebetween; the distal end of the hollow sleeve further includes an open sleeve lip; the cannula further includes an inflatable balloon assembly, a one-way valve assembly for inflation and deflation, and a gas access connecting the balloon assembly and the one-way valve assembly; the balloon assembly includes a balloon lip and an balloon body coupled thereto; the cannula is mounted on an outer portion of the hollow sleeve; the balloon lip is wrapped on the exterior surface of the sleeve lip; the balloon assembly further includes a movable mechanism which are moved from the distal end to the proximal end to cause the balloon body stretched from the free state to the straight state when the cannula is inserted or removed; when the puncture is completed, operating the movable mechanism from the proximal end to the distal end to return the balloon body from the straight state to the free state, and then the gas is injected through the gas access to inflate the balloon body.

In an optional solution, the movable mechanism includes the outer sleeve extending proximally from the balloon body, and the slip tube wrapped by the outer sleeve; the outer sleeve and the slip tube are aligned at the distal end, the outer sleeve with the axial length Lo, the slip tube with the axial length Li, and Lo>Li.

In an optional solution, the sleeve lip including a slanted cylinder surface between the opening lip and the transitional lip, the inner cylinder surface and the slanted cylinder surface limiting the sleeve slanted-wall; the balloon lip extending distally forms the balloon slanted-wall that matches the shape and size of the sleeve slanted-wall; the balloon lip extends proximally to the balloon body connected and smoothly transitioned thereof; the balloon slanted-wall and the sleeve slanted-wall form a taper fit; and the balloon slanted-wall and the sleeve slanted-wall are fixed to form an annular or curved complete-closed taper seam region to connect the balloon lip and the sleeve lip into a whole.

In an optional solution, both the balloon lip and the balloon body have the uniform thickness Ta3, and 0.05 [symbol] Ta3 [symbol] 0.1 mm.

In an optional solution, the balloon body comprises the balloon proximal transition region, the balloon distal transition region, and the balloon body extending therebetween. The balloon lip is seamlessly coupled and smoothly transitioned to the balloon distal transition region. The outer sleeve comprises the outer-shaft distal-end and the outer-shaft proximal-end and the outer-shaft wall extending therebetween. The outer-shaft distal-end is connected to the balloon proximal transition region as a whole.

In an optional solution, the balloon plane of the balloon body and the balloon axis forms the included angle Aballoon, and 45°≤Aballoon≤85°.

In an optional solution, the hollow sleeve includes the integral wedge-shaped opening lip, the opening lip limiting the mouth plane, and the angle Aopen which is formed by the mouth plane intersects the axis, and 45°≤Aopen≤85°.

In an optional solution, the cannula further includes an external fixation assembly that includes a gasket and a lock member disposed along the axis. The gasket includes the gasket distal-end thereof, the acute angle Aclamp which is formed by the gasket distal-end intersects the axis, and Aclamp=Aopen.

In an optional solution, the outer-shaft proximal-end is fixed to the exterior surface of the outer cylinder surface and the valve seat of the one-way valve assembly to form a fully closed proximal seam region. The gas access connecting the cannula and the one-way valve assembly is formed between the outer sleeve and the outer cylinder surface.

In an optional solution, the slanted cylinder surface and the outer cylinder surface forms the included angle Alip, wherein 3°≤Alip≤15°.

### BRIEF DESCRIPTION OF THE DRAWINGS

A more complete appreciation of this invention, and many of the attendant advantages thereof will be readily apparent as the same becomes better understood by reference to the following detailed description, wherein:

FIG. 1 is a perspective view of the trocar 5 in the prior art;

FIG. 2 is a partial enlarged schematic view of the distal end portion of the trocar 5 in FIG. 1;

FIG. 3 is a partial section view of the distal end portion of the trocar 5 in FIG. 2;

FIG. 4 is a 3D perspective view of the trocar 7 in the prior art;

FIG. 5 is a partial cross-sectional view of the distal end portion of the inner sleeve 54 in the prior art;

FIG. 6 is a partial cross-sectional view of the distal end portion of the outer sleeve 55 in the prior art;

FIG. 7 is a partial enlarged schematic view of the distal end portion of the trocar 7 in FIG. 4;

FIG. 8 is a partial cross-sectional of the distal end portion of the trocar 7 in FIG. 7;

FIG. 9 is a 3D perspective view of the cannula 100 in the present invention;

FIG. 10 is an exploded view of the cannula 100 in FIG. 9;

FIG. 11 is a 3D perspective view of the lower chamber 123 in FIG. 10;

FIG. 12 is an axial section view of the lower chamber 123 in FIG. 11;

FIG. 13 is a partial enlarged view of the distal end portion of the lower chamber 123 in FIG. 12;

FIG. 14 is an axial section view of the balloon assembly 240 in FIG. 10;

FIG. 15 is an enlarged view of the distal end portion of the balloon assembly 120 in FIG. 14;

FIG. 16 is a sectional view along line 16-16 in FIG. 18;

FIG. 17 is an axial section view of the second seal assembly 120 in FIG. 9;

FIG. 18 is an enlarged view of the distal end portion of the assembly 120 in FIG. 17;

FIG. 19 is a partial enlarged schematic view of the distal end portion of the one-way valve assembly 140 in FIG. 17;

FIG. 20 is a sectional view along line 20-20 in FIG. 19;

FIG. 21 is an exploded view of the external fixation assembly 160;

FIG. 22 is a 3D perspective view of the fixed assembly after assembled in FIG. 21;

FIG. 23 is a sectional view of the fixed assembly in FIG. 21;

FIG. 24 is a 3D perspective view of the cannula 100 of the external fixation assembly;

FIG. 25 is a schematic view showing the balloon body 270 of the cannula assembly in a stretched state in FIG. 24;

FIG. 26 is a simulation view of the cannula puncturing the body wall of the patient in FIG. 25;

FIG. 27 is a schematic simulation view of the cannula secured to the body wall of the patient in FIG. 24;

FIG. 28 is a 3D perspective view of the cannula 300 in the present invention;

FIG. 29 is an exploded view of the cannula 300 in FIG. 28;

FIG. 30 is a 3D perspective view of the lower chamber 323 in FIG. 29;

FIG. 31 is an axial section view of the lower chamber 323 in FIG. 30;

FIG. 32 is a partial enlarged view of the distal end portion of the lower chamber 323 in FIG. 31;

FIG. 33 is an axial section view of the balloon assembly 340 in FIG. 29;

FIG. 34 is an enlarged view of the distal end portion of the balloon assembly 340 in FIG. 33;

FIG. 35 is an axial section view of the second seal assembly 120a in FIG. 29;

FIG. 36 is an enlarged view of the distal end portion of the assembly 120a in FIG. 35;

FIG. 37 is a schematic view of the cannula 300 of the external fixation assembly 160a;

FIG. 38 is a simulation view of the cannula puncturing the body wall of the patient in FIG. 37;

In all views, the same referred number shows the same element or assembly.

### DETAILED DESCRIPTION OF EMBODIMENTS

Embodiments of the invention are disclosed herein, however, it should be understood that the disclosed embodiments are merely examples of the invention, which may be implemented in different ways. Therefore, the invention is not intended to be limited to the detail shown, rather, it is only considered as the basis of the claims and the basis for teaching those skilled in the art how to use the invention. For convenience of description, the position that is close to the operator is defined as the proximal end, and the position far from the operator is defined as the distal end. Where the axis direction of the shaft portion of the obturator or the sleeve axis direction of the cannula is defined as the axial direction, and the direction substantially perpendicular to the axial direction is defined as the transverse direction.

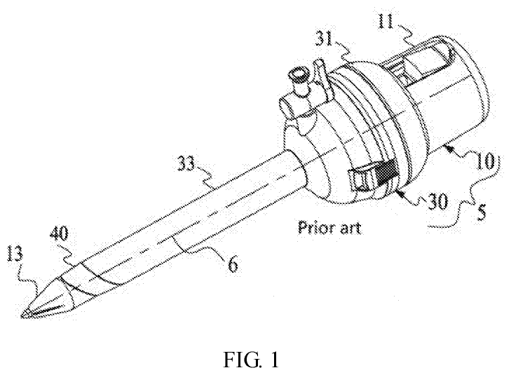

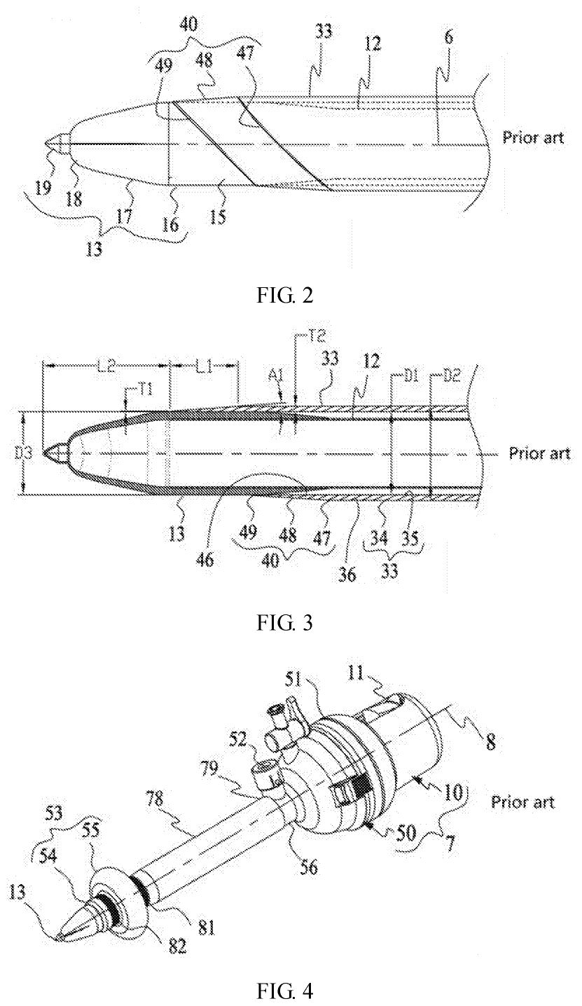

Those skilled in the art will understand that the trocar typically includes a cannula and a puncture needle, the cannula including the instrument seal, the duckbill seal and the hollow sleeve. For example, referring to CN201610630336.5 herein, the invention of which is entitled “Trocar Seal System Capable of Seal”, the cannula disclosed in the application of Chinese Patent filed on Aug. 2, 2016. The obturator is composed of a handle, a shaft and the distal-end portion. For example, referring to CN201611125444.3 herein, the invention of which is entitled “An Improved Transparent Bladeless Obturator”, the obturator disclosed in the application of Chinese Patent filed on Dec. 9, 2016. Referring to FIGS. 1-3, the trocar 5 for the direct puncture technique in the prior art includes an obturator 10, a cannula 30 and an axis 6. The obturator 10 comprises a handle 11, a transparent tip 13 and a shaft 12 therebetween. From the distal end to the proximal end, said transparent tip 13 is divided into a top-portion 19, a spear-portion 18, a transition-portion 17 and a base-portion 16. The top-portion 19, a spear-portion 18, a transition-portion 17 and a base-portion 16 are sequentially connected, wherein the connection of the top-portion 19 and the spear-portion 18 may be not smooth. Because the shape and size of the top-portion and the spear-portion are relatively small and sharp, the obturator 10 is mainly for puncturing and separating during the process of puncturing the muscle or tissue of the patient. While the spear-portion 18, the connection between the transition-portion 17 and the base-portion 16 is generally a streamlined smooth transition to facilitate squeezing and inflating the wound and reducing the puncture force. At the same time, the base-portion 16 further includes a cylinder-portion 15 that matches the size of the inner-aperture of the sleeve 32 of the cannula 30, which is advantageous that the obturator and the cannula form the smooth transition and advantageous to reduce the required puncture force of the cannula assembly 30 to squeeze and inflate the wound.

Referring to FIGS. 1-3, the cannula 30 includes a seal assembly 31 and a sleeve lip 40 and the hollow sleeve 33 extending therebetween. The sleeve 33 includes the inner cylinder surface 35 with the inner diameter D1 and the outer cylinder surface 34 with the outer diameter D2 and the sleeve-wall portion 36 therebetween. A 12 mm diameter cannula 30, wherein D1=12.8 mm, D2=14.6 mm. The sleeve lip 40 includes a slanted cylinder surface 48 between the opening lip 49 and the transition lip 47, the inner cylindrical surface 35 and the slanted cylinder surface 48 limiting the slanted wall portion 46, which of the slanted wall portion 46 at the position of the opening lip is T1, the wall thickness at the position of the transition lip is T2, and the wall thickness of the sleeve wall portion 36 is approximately equal to T2; the thickness of the slanted wall portion 46 gradually increases from the distal end to the proximal end, and generally the increasing rate of the thickness is small, resulting in the smoother transition between the obturator 10 and the cannula 30. Referring to FIG. 3, in a typical design, 0.1 mm [symbol] T1 [symbol] 0.3 mm, 0.8 mm [symbol] T2 [symbol] 1.1 mm, and the angle A1 between the slanted cylinder surface 48 and the inner cylinder surface 35, 3° [symbol] A1 [symbol] 15°; the length L1 of the slanted wall portion 46 along the axial direction, 6 mm [symbol] L1 [symbol] 12 mm.

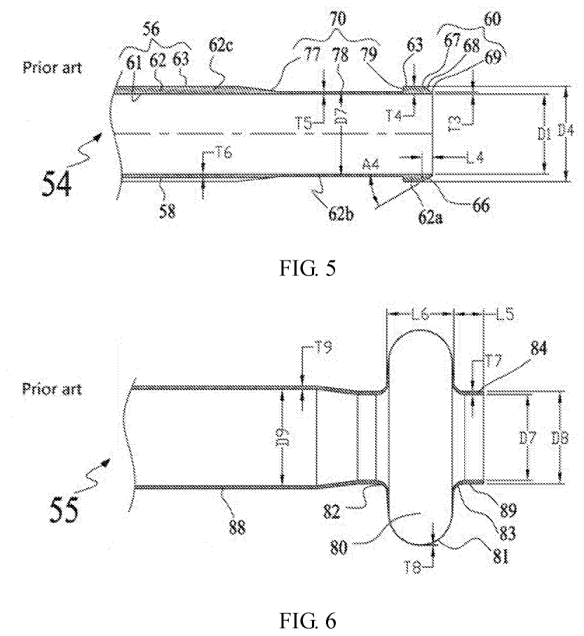

The balloon assembly comprising a balloon of an elastomeric material disclosed in, for example, U.S. Pat. Nos. 5,468,248 and 6,904,454, which is generally a double-layer sleeve and secured by glue bonding. For the transition of the sleeve lip is not smooth, it is usually not used in the field of the direct puncture technique. For example, the cannula comprising the balloon with a non-elastic material disclosed in U.S. Pat. No. 8,888,692 (abbr. P692) has a smaller outer diameter to help reduce the puncture force during puncturing. The balloon cannula disclosed herein and based on patent P692 is referenced herein. FIGS. 4-8 depict a balloon-containing trocar 7 substantially identical to the trocar comprising the balloon disclosed in the patent P692. Briefly, the trocar 7 includes the obturator 10, the cannula comprising the balloon 50 and the axis 8. The cannula comprising the balloon 50 includes the seal assembly 51 and the sleeve 53 connected thereto and extending to the distal end, the sleeve 53 including the inner sleeve 54 and the outer sleeve 55. Referring to FIG. 5, the inner sleeve 54 includes the sleeve lip 60 and a hollow shaft 56 that connects the seal assembly 51. The hollow shaft 56 includes the inner cylinder surface 61 with the inner diameter D1 and the outer-shaft cylinder surface 63 with the outer diameter D4 and a sleeve wall portion 62 therebetween. The sleeve lip 60 includes a slanted cylinder surface 68 between the opening lip 69 and the transition lip 67, the inner cylindrical surface 61 and the slanted cylinder surface 68 limiting the slanted wall portion 66. The hollow shaft 56 is adjacent the annular groove 70 of the sleeve lip 60, and the annular groove 70 includes a stepped surface 79 and a transition surface 77 and a grooved surface 78 with the diameter D7 therebetween. The groove 70 divides the sleeve-wall portion 62 into a sleeve distal-wall 62a, a sleeve groove-wall 62b and a sleeve proximal-wall 62c. The adjacent region which is the connection position of the hollow shaft 56 with the seal assembly 51 includes a one-way valve assembly 52 along which the passageway 58 extends and connects the groove 70 with the one-way valve assembly 52. With continued referring to FIG. 5, in a typical design, the thickness of the slanted wall-portion 66 at the position of the opening lip is T3 and the thickness at the transition lip portion is T4, and the thickness of the sleeve groove wall 62b is T5, wherein 0.1 mm [symbol] T3 [symbol] 0.3 mm, 1.15 mm [symbol] T4 [symbol] 1.25 mm, and 0.6 mm [symbol] T5 [symbol] 5 0.8 mm. The angle between the slanted cylinder surface 68 and the inner cylinder surface 61 is A4, 25° [symbol] A1 [symbol] 45; the length L4 of the slanted wall portion 66 along the axial direction, 2 mm [symbol] L1 [symbol] 5 mm.

Referring now to FIG. 6, the outer sleeve 55 includes an outer-shaft distal-end 89 and an outer tube proximal end 87 and a hollow shaft 88 therebetween. The balloon 80 is disposed in the adjacent region of the outer tube distal-end 89, wherein the balloon 80 includes a balloon-body 81, and a balloon distal transition region 83 extending distally from the balloon body 81 and coupled to the sleeve distal end 89; A balloon proximal transition region 82 extending proximally from the balloon-body 81 and coupled to the hollow shaft 88. The balloon distal-end 89 includes a cylindrical wall 84 with the inner diameter D7, the outer diameter D8, and the thickness T7. The thickness T8 of the balloon body 81 is smaller than the thickness T7, and the thickness of the transition region 83 (82) is changed from T7 to T8. In a typical design and manufacturing solution, the outer sleeve 55 is manufactured by stretch blow molding, wherein 0.2 mm [symbol] T7 [symbol] 0.3 mm, 0.01 mm [symbol] T8 [symbol] 0.05 mm, and D8<D4.

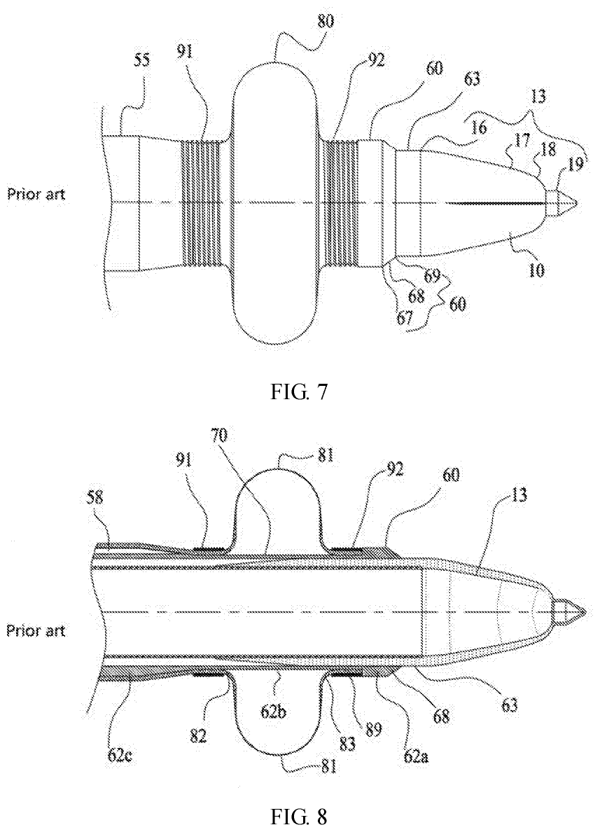

FIGS. 7-8 depict a schematic view of the distal end of the inner sleeve 54 and the outer sleeves 55. Wherein the outer-shaft distal-end 89 and the balloon 80 are mounted at the annular groove 70, wherein the outer-shaft distal-end 89 mates with the groove surface 78 and the stepped surface 79. The cord coil 91 and the cord coil 92 respectively fix the balloon proximal transition region 82, the balloon distal transition region 83 and the outer-shaft proximal-end 89 to the groove 70 and are sealed with glue. Typically, the size after the cord coil 92 is wound by the outer-shaft distal-end 89 is still less than or equal to the maximum outer diameter D4 of the sleeve lip 60. With FIGS. 3 and 8, the distal end of the trocar 5, the transition the obturator 10 and the cannula 30 is smooth; and the transition of the distal end of the trocar 7, the obturator 10 and the cannula 50 is not smooth. It mainly includes two transition regions which are not smooth, one of which is a sudden change in size between the transparent tip 13 and the sleeve lip 60, resulting in a non-smooth transition; the other of which is that the transition of the balloon itself is not smooth, particularly, the thickness of the distal-end transition region 83 of the balloon is not uniform due to uneven thickness and irregular shape. It will be appreciated by one of ordinary skill that the size variation between the transparent tip 13 and the sleeve lip 60 can be reduced by reducing the included angle A4, however reducing the included angle A4 while ensuring the annular groove 70 has sufficient strength while ensuring that D8<D4, and the length L4 of the slanted wall portion 66 along the axial direction should be equal or greater than 10 mm. The size of the balloon body 81 and the outer-shaft distal-end 89 in the axial direction is usually equal or greater than 20 mm. Those skilled in the art will appreciate that due to the abdominal space limitation of the patient, the length of the distal-end of the cannula 30 or 50 puncturing the abdominal wall into the abdominal cavity usually does not exceed 20 mm, otherwise it may affect the operation of the surgery or cause accidental injury to the internal organs during the puncture, so it is impossible to extend L4 to reduce the transition change.

The effect of the transitional change on the puncture force is quite large: for example, the 12 mm-size balloon cannula disclosed in Patent P692 (without folding the balloon) passes through the tissue incision, and when the shaft without the balloon passes, the resistance is substantially zero or small. When the balloon passes through the incision, its peak resistance reaches 25 pounds; and when the balloon folding technique is used, its peak resistance drops to 13 pounds. This is mainly because the regular folding of the balloon can reduce the structural size change caused by the random accumulation of the balloon body during the puncture, thereby contributing to the large reduction of the puncture resistance. Those skilled in the art will appreciate that the difference in resistance caused by the difference in the size of the flexible balloon body is already large, and the increase in resistance caused by the sudden change in rigidity between the transparent tip 13 and the sleeve lip 60 will be more pronounced. The puncture force (the direct puncture technique) of a 12-mm visual obturator that penetrates the abdominal wall of the patient is disclosed in US Patent Application No. 20070066988A1, which is approximately 15 pound. Normally less than 15 pounds of the puncture force is more comfortable during the operation, while above 18 pounds will generally affect the surgeon's control during puncturing. Obviously, if the resistance of the balloon change is included, and the resistance caused by the sudden change between the transparent tip 13 and the sleeve lip 60, the trocar 7 cannot be applied to the direct puncture technique.

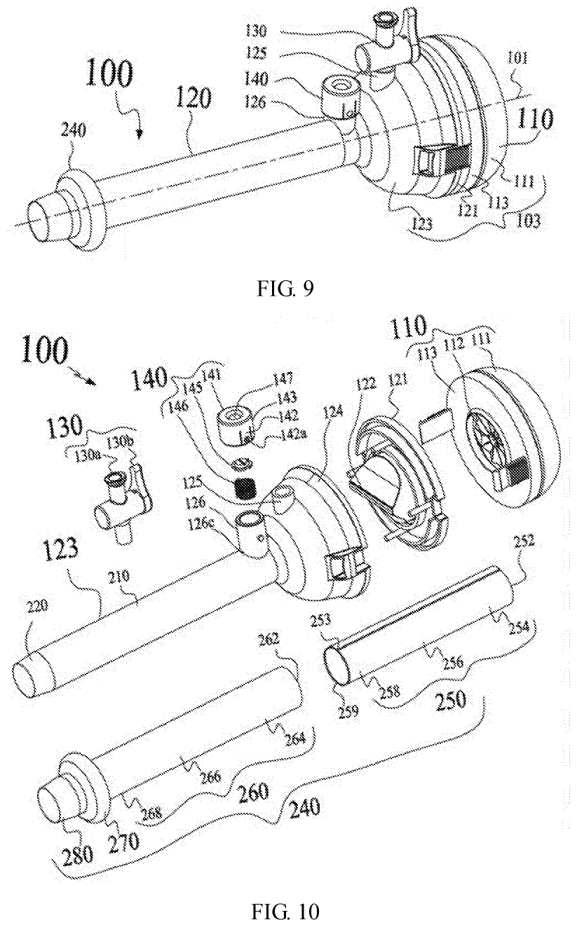

FIGS. 9-10 depict the cannula 100 in the first embodiment of the present invention. The cannula 100 includes an axis 101 and the first seal assembly 110 and the second seal assembly 120 that are axially placed. The first seal assembly 100 includes an instrument seal 112 sandwiched between the upper chamber 111 and the upper cover 113. The second seal assembly includes the duck bill 122 sandwiched between the lower cover 121 and the lower chamber 123. The upper cover 113, the upper cover 117, the lower cover body 121 and the lower chamber 123 are sequentially connected to form the outer casing 103. The outer casing 103, the instrument seal 112 and the duck bill 122 constitute the seal system that includes substantially aligned apertures. The instrument seal 112 tightens the instrument and forms a seal when the external instrument is inserted into the cannula 100; the duck bill 122 typically does not provide a seal for the instrument inserted, but automatically closes and forms a seal when the instrument is removed. In order to save space, the detailed depiction and presentation of the connecting and fixing manner and the structure of the upper casing 111, the instrument seal 112, the upper cover 113, the lower cover 121, the duck bill 122 are omitted. The above structure can be understood in combined with the related description disclosed in the aforementioned application Chinese invention application CN201610630336.5. Those skilled in the art will appreciate that in the prior art disclosed, the instrument seal 112 and the duck bill 122 are implemented in a variety of ways. For example, the four-overlapping-segment seal assembly disclosed in U.S. Pat. No. 8,029,475, pleated trocar seal disclosed in U.S. Pat. No.6,482,181, duck bill with four fold sections disclosed in U.S. Pat. No. 5,443,452, multi-angled duckbill seal assembly disclosed in U.S. Pat. No.8,034,032. For other disclosed seal assembly, the duck bill and the outer casing can be modified slightly to replace the seal assembly, duck bill, the upper chamber, the upper cover, the lower cover, etc.

With continued referring to FIGS. 9-10, the lower chamber 123 also includes a lower outer casing 124 and a hollow sleeve 210 coupled thereto and extending distally. The lower chamber 123 also includes a balloon assembly 240, a valve assembly 130 and a one-way valve assembly 140. The gas valve assembly 130 includes a gas valve body 130a and a gas valve core 130b that is inserted into the gas valve body 130a and transversely penetrates the valve mounting hole 125 of the lower outer casing 124 together.

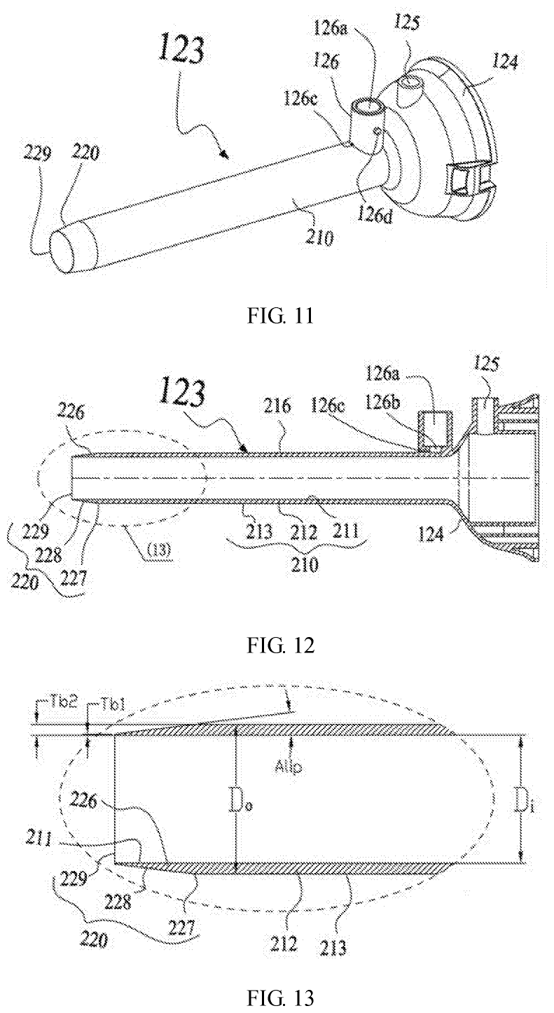

Referring to FIGS. 11-13, the hollow sleeve 210 includes an inner cylindrical surface 211 having an inner diameter Di and an outer cylinder surface 213 with the outer diameter Do and a sleeve wall 212 with the thickness Ta1 therebetween; the distal end of the hollow sleeve 210 also includes an open sleeve lip 220 including a slanted cylinder surface 227 between the opening lip 229 and the transitional lip 228, the inner cylinder surface 211 and the slanted cylinder surface 227 limiting the sleeve slanted-wall 226, which has the thickness Tb1 at the opening lip 229 and the thickness Tb2 at the transition lip 228. The thickness of the slanted wall portion 226 gradually increases from the distal end to the proximal end, and generally the increase rate of the thickness is small, resulting in the smoother transition between the obturator and the cannula. In a preferred solution, Tb2=Ta, and 0.1 mm [symbol] Tb1 [symbol] 0.3 mm, and 0.8 mm [symbol] Tb1 [symbol] 1.1 mm. Generally, it is difficult to manufacture when Tb1<0.1 mm and Tb1>0.3 mm will result in the transition is more seriously not smooth. Generally, when Tb2<0.8 mm, the strength of the hollow sleeve 210 is not enough, and when the Tb2>1.1 mm, the outer diameter of the hollow sleeve 210 is too large to be advantageous to minimize the damage to the patient. In another preferred solution, the angle Alip between the slanted cylindrical surface 227 and the inner cylindrical surface 211, wherein 3 [symbol] Alip [symbol] 15°. The Alip <3° results in insufficient strength of the sleeve lip 220 which is difficult to manufacture, while the Alip>15° results in a non-smooth transition between the obturator and the cannula.

With continued reference to FIGS. 11-13, the proximal end of the hollow sleeve 210 includes a valve seat 126 for mounting the one-way valve assembly 140. The valve seat 126 includes a mounting hole 126a and a counterbore 126b limited by a cylindrical side-wall. The valve seat 126 further includes the side-hole 126c that penetrates through the cylindrical side wall and communicates with the counterbore 126b.

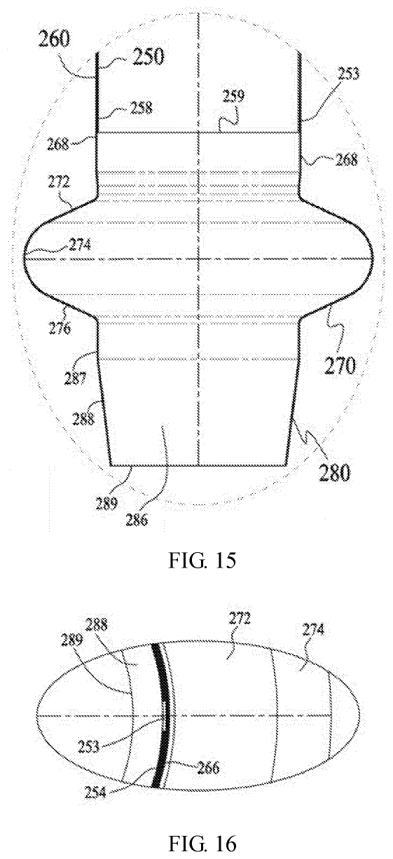

Referring to FIGS. 10 and 14-16, the balloon assembly 240 includes the flexible outer sleeve 260 and the balloon bag lip 280, the balloon body 270 therebetween and the rigid slip tube 250. The balloon body 270 includes the balloon proximal transition region 272, the balloon distal transition region 276 and the balloon body 274 extending therebetween. The balloon lip 280 includes the balloon opening lip 289, the balloon transition lip 287 and the balloon slanted-wall 288 extending therebetween, the balloon slanted-wall 288 limiting the conical bore 286 that matches the shape and size of the sleeve slanted-wall 226. The balloon transition lip 287 extends proximally from the distal end and is seamlessly coupled and smoothly transitioned to the balloon distal transition region 276. The outer sleeve 260 includes the outer-shaft distal-end 268, the outer-shaft proximal-end 264 and the outer-shaft wall 266 extending therebetween, the outer-shaft distal-end 268 being seamlessly coupled and smoothly transitioned to the balloon proximal transition region 272. The outer-shaft wall 266 limits a cylindrical bore 265 that conforms to the shape and size of the outer cylinder surface 213, the outer-shaft proximal-end 264 limiting an outer-shaft opening 262. In a preferred solution, the balloon lip 280 and the balloon body 270 have a substantially uniform thickness Ta3. In an optional solution, 0.05mm [symbol] Ta3 [symbol] 0.1 mm, when the thickness of the balloon body is less than 0.05 mm, the balloon body is difficult to manufacture and the strength is insufficient to cause the rupture failure. When the thickness of the balloon body is greater than 0.1 mm, the thickness of the material which is folded and accumulated after being evacuated is increased to a large extent, and the puncture force is increased.

The slip tube 250 includes the slip-tube distal-end 258, the slip-tube proximal-end 254 and the slip-tube wall 256 extending therebetween. The slip-tube distal-end 258 limits a slip-tube distal opening 259, and the slip-tube proximal end 254 limits the slip-tube proximal opening 252. The slip tube 250 further includes a cutting groove 253 extending therethrough, the cutting groove 253 cutting the slip-tube wall 256 into the open tube-shape with the non-closed circular cross-section. The outer sleeve 260 has the axial length Lo, the slip tube 250 has the axial length Li, and Lo>Li.

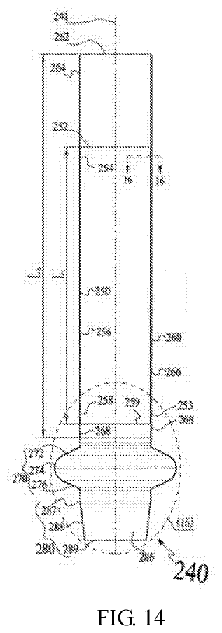

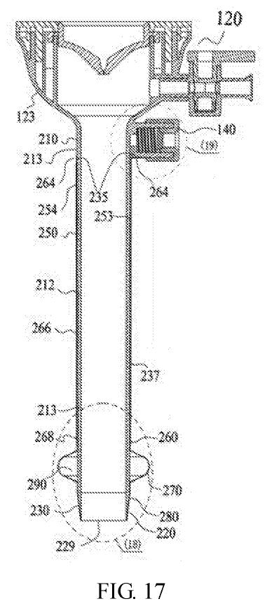

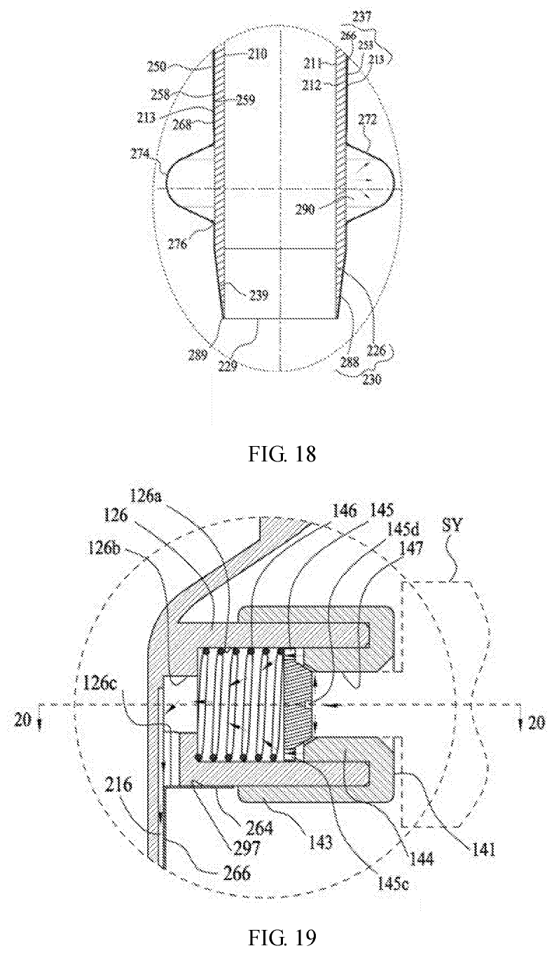

Referring to FIGS. 14-16, the sliping outer tube 250 is mounted inside the outer sleeve 260, wherein the slip-tube distal end 258 is substantially aligned with the outer-shaft distal-end 268, and the outer-shaft proximal-end 264 extends beyond the slip-tube proximal-end 254 continues to extend to the proximal end. Preferably, the slip-tub wall 256 and the outer-shaft wall 266 are all or most fixed together by glue bonding to form the balloon assembly 240. Referring to FIGS. 17-18, the balloon assembly 240 is mounted on the exterior of the hollow sleeve 210, wherein the balloon lip 280 is wrapped around the exterior surface of the sleeve lip 220; all of the slip tube 250 and the proximal end portion of the outer sleeve 260 are wrapped around the exterior of the outer cylinder surface 213. The balloon slanted-wall 288 mates with the sleeve slanted-wall 226 to form a taper fit 230. In one solution, the annular fully closed taper seam region 239 is formed between the balloon slanted-wall 288 and the sleeve slanted-wall 226 by welding (or glue bonding), thereby connecting the balloon lip 280 and the sleeve lip 220 into a whole. The outer-shaft proximal end 264 is bonded to the outer cylinder surface 213 and the exterior surface of the valve seat 126 to form a fully closed proximal seam region 235. The outer cylinder surface 213, the cutting groove 253, and the outer-shaft wall 266 constitute the passageway 237 that communicates with the one-way valve assembly 140 and the balloon body 270. The one-way valve assembly 140, the passageway 237, the outer cylinder surface 213, the balloon assembly 240, the proximal seam region 235 and the taper seam region 239 form a closed balloon cavity 290.

Referring to FIGS. 10, 17, 19 and 20, the one-way valve assembly 140 includes a bonnet 141, a one-way plug 145, a spring 146 and a valve seat 126. The bonnet 141 includes a gas hole 147 therethrough, an inner wall 144 that limits the gas hole 147, and an outer wall 143. The inner wall 144 and the outer wall 143 form an annular groove and are mated with a mounting hole 126a limited by the valve seat 126, and the mounting hole 126a communicates with the side-hole 126c. The outer wall 143 includes an elastic arm 142, and the distal end of the elastic arm 142 is disposed with a limit-hole 142a the size and position of which match the limit-pin 126d outside the valve seat 126. When the one-way valve assembly 140 is installed, the spring 146 is first placed in the valve seat 126, then the one-way plug 145 is inserted and the bonnet 140 is snapped onto the valve seat 126. After the elastic arm 142 is deformed, the limit-pin 126d enters into the limit-hole 142a and compresses the spring 146. Due to the counterforce of the spring 146, the one-way plug 145 is pushed outwardly against the inner wall 144 of the bonnet 140 to form a seal.

In one solution, the inflation or deflation is performed by the one-way valve assembly 140 using a standard syringe. Referring to FIGS. 18-19, the gas hole 147 are sized to match the shape and size of a standard syringe tip. The distal end of the gas hole 147 includes a conical hole 147b, and the one-way plug 145 includes a cone 145b with the taper fit thereof and a planar wall 145a extending distally. The planar wall 145a includes a vent groove 145c and the cone 145b includes a flat or cross venting groove 145d. In the natural state, the counterforce of the spring 146 pushes the one-way plug 145 outward, and the cone 145b matches the conical bore 147b to form a seal, preventing gas leakage in the balloon body-cavity 290.

FIGS. 16-19 depict the inflation process of the balloon assembly 240. Specifically, the syringe SY mouth is inserted into the gas hole 147 of the one-way valve assembly 140, and the one-way plug 145 is opened inwardly, and then the gas is injected. The gas passes through the venting groove 145c and the venting groove 145d of the one-way plug 145, and then enters the valve seat 126 through the clearance of the inner wall 144 of the bonnet 140, through the counterbore 126b, the side-hole 126c, and the passageway 216 into the balloon body 270 which is then inflated. Removed of the syringe SY, the counterforce of the spring 146 pushes the one-way plug 145 outwardly, and the cone 145b matches the conical bore 147b to form a seal to prevent gas leakage.

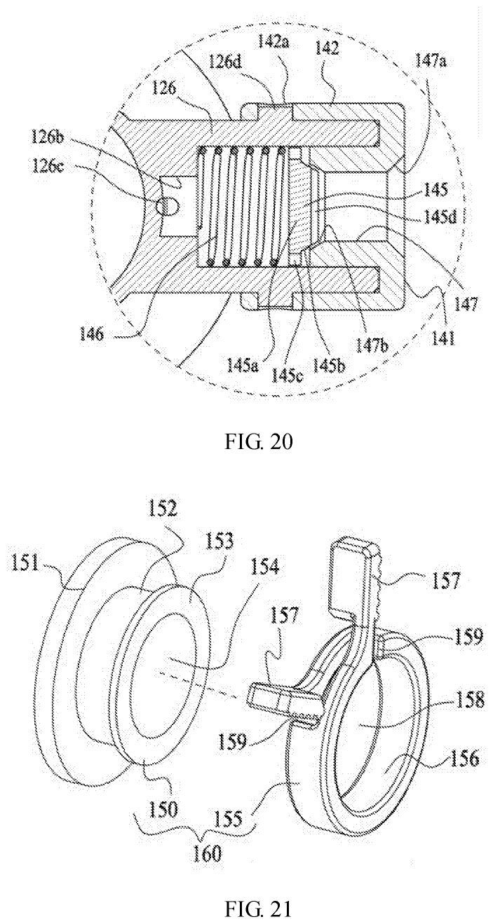

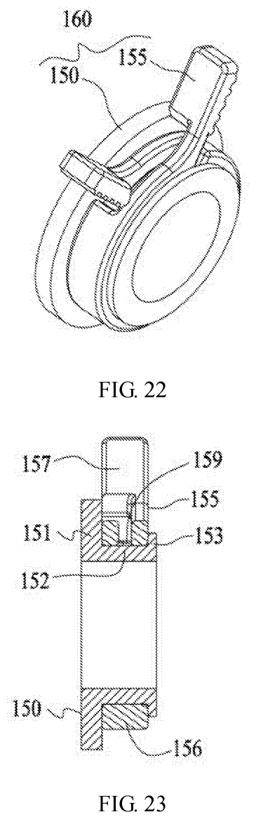

As shown in FIGS. 20-23, the cannula 100 further includes an external fixation assembly 160 that includes a gasket 150 and a lock member 155. The gasket 150 is made of a flexible material, including but not limited to, rubber, sponge, and so on. The gasket 150 includes the distal end 151 and the proximal end 153 thereof, and a ring-groove 152 extending therebetween. The aperture 154 extending through the gasket 150, the diameter of the aperture 154 smaller than the outer diameter of the outer sleeve 260, can be inserted into the outer sleeve 260 by inflation. The distal end 151 is placed around the incision of the abdominal wall to protect the incision from leakage of the pressure from the incision site. The lock member 155 is made of plastic with good elasticity (for example, polycarbonate) or metal (for example, SUS301). The lock member 155 includes a lock body 156, a handle 157 and a limit edge 159 extending from the opposite ends of the lock body 156, which forms a lockhole 18 by prefabricated crimping, and the lock member 155 forms a locking force that crimps inwardly. The inward crimping force of the lock body 156 stagger-limits the handle 157 at both ends of the lock body 156 and the limit edge 159, and the two handles 157 are staggered to form an approximately V-shape. The lockhole 158 can be enlarged or reduced by pinching or releasing the two handles 157. FIGS. 22-24, the locking member 155 is fitted into the ring-groove 152 of the gasket 150. Since the locking member 155 forms the locking force of the inward crimping, the locking member 155 will gasket 150 in the released state of the lock member 155, the lock member 155 locks the gasket 150 and generates an inward holding force to the hole 154.

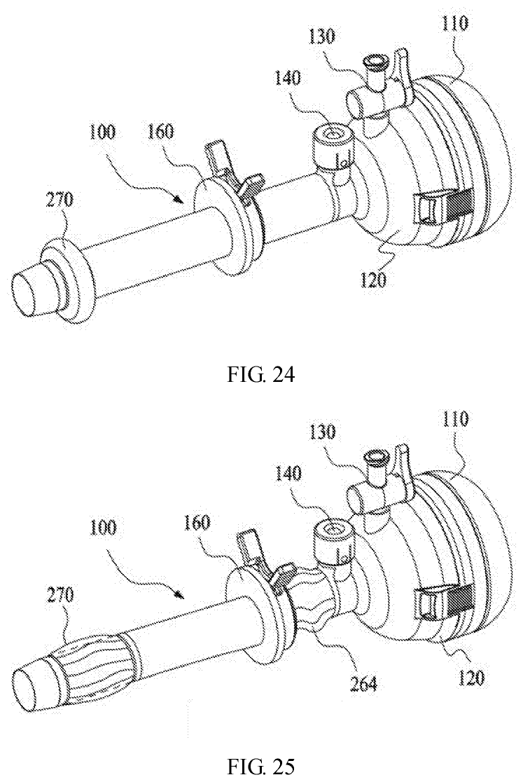

Referring to FIGS. 24 and 27, the external fixation assembly 160 is mounted at the proximal end position at the exterior of the outer sleeve 260. The cannula is inserted into the body cavity through the abdominal wall of the patient as an access for the instrument to get in or out the body cavity. When the surgeon operates various instruments, such as grasping forceps, shears, anastomat, etc., the contact between the surgical instrument and the cannula creates the friction that may cause the cannula to move inward or outward along the abdominal wall. If the cannula is not fixed, it may result in slipping out of the abdominal wall or further into the interior of the body cavity, so it is extremely important that the cannula is firmly fixed to the abdominal wall. The balloon body 270 of the cannula 100 is inside the body cavity of the patient and is inflated. Adjusting the position of the external fixation assembly 160 along the axial direction of the cannula 100 for the abdominal wall is tightened between the balloon body 270 and the external fixation assembly 160, thereby the cannula 100 is securely fixed over the abdominal wall to simultaneously prevent the cannula 100 from moving in or out of the body. It will be conceived by those skilled in the art that the fixing mechanism or clip disclosed in the prior art, such as the U.S. Pat. Nos. 7,300,448, 7,316,699, 7,691,089, 8,162,893 can be modified slightly to replace the external fixation assembly 160 of the present invention. Other solutions are also conceivable.

In connection with the background art and the foregoing description of the cannula 50 in the prior art , the thickness T7 of the cylindrical wall 84, wherein 0.2 mm [symbol] T7 [symbol] 0.3 mm; the thickness T8 of the balloon body 81, wherein 0.01 mm [symbol] T8 [symbol] 0.05 mm; the thickness of the transition region 83 (82) is gradually changed from T7 to T8. However, when the thickness of the balloon body is 0.01-0.05 mm, the manufacture of the balloon body is often complicated and difficult to control, or it is expressed as that the strict precision control of the thickness increases the manufacturing cost. he balloon body 81 during using is born a relatively high pressure (about 25 Psi-30 Psi), and the thickness of the balloon body 81 is designed to be thin and the thickness of the cylindrical wall 84 is about 10 times than it, so the design is unreasonable in terms of the structural strength. Moreover, it is difficult to avoid to form the thickness uneven in the transition region of the balloon by the stretch blow molding for manufacturing the balloon disclosed in the Patent P692. The thickness of the outer-shaft distal end 89 and the hollow shaft 88 is thick, and the thickness of the transition portion 83 (82) is uneven. The thickness of the material overlapped after being evacuated greatly increases, and the puncture force is increased to a greater extent. Additionally, the sleeve lip 60 of the cannula 50 causes the uneven transitional between the cannula and the obturator to further increase the puncture force. Therefore, the disclosed and commercialized cannula with the balloon to date are generally not used in the direct puncture technique.

The cannula 100 disclosed herein has a sleeve lip 220 that is substantially identical to the cannula without balloon for the direct puncture technique disclosed in the prior art; the balloon assembly 240 includes a slanted balloon lip 280 that mates with the sleeve lip 220; moreover, the balloon lip 280 and the sleeve lip 220 are fixed by welding (or bonding) to reduce a sudden change in the size of the mating position. In addition, the thickness Ta3 of the balloon lip 280 and the balloon body 270 of the balloon assembly 240 in the present invention is substantially uniform and 0.05 mm [symbol] Ta3 [symbol] 0.1 mm, which enhances the strength of the balloon and reduces the difficulty of controlling the thickness, and at the same time reduces the deformation force of the transition region caused by the uneven thickness of the material, so that the cannula 100 in the present invention can be used in the direct puncture technique. Of course, the cannula 100 in the present invention can also be used in the Hasson technique.

When the balloon lip 280 and the sleeve lip 220 are fixed by glue bonding, UV-curing adhesive that meets biocompatibility requirements is preferred. UV-curing adhesive has a fast curing speed and a strong bonding ability, and the thinner glue layer can achieve the desired fixing strength, thereby reducing the size change of the bonding position. While, other glues suitable for biocompatibility, such as epoxy resin, polyester glue, etc., may also be selected depending on the material of the sleeve and the balloon body. When the balloon lip 280 and the sleeve lip 220 are fixed by welding, there are many types of welding techniques, including but not limited to thermal compression welding, ultrasonic welding, high frequency welding, radiation welding, pulse welding, etc. Compared with the glue bonding, the welding is superior. Welding enables the balloon lip 280 and the sleeve lip 220 to be connected reliably and transition smoothly (a smooth transition can be achieved even after trimming the residual side of the welding), and the thickness of the new sleeve lip formed by the welding is smaller than the sum of the thicknesses of the balloon lip 280 and the sleeve lip 220.

Those skilled in the art will understand that it is critical that the balloon slanted-wall 288 be mated with the sleeve slanted-wall 226 to form a taper fit 230. It is beneficial to improve the assembly efficiency and the fastness when the balloon lip 280 and the sleeve lip 220 are fixed to each other. Particularly for welding fixation, the taper fit 230 ensures that the welding head can be opened and closed and the holder can be supported along the axial direction of the sleeve assembly during welding, so that the taper seam region 239 can be formed by one-time welding. In the case of a non-tapered fit, it is often necessary to weld to form a circular closed seam, which often requires multiple times of welding, which is likely to cause local accidental thinning of the balloon body to cause root cutting failure.

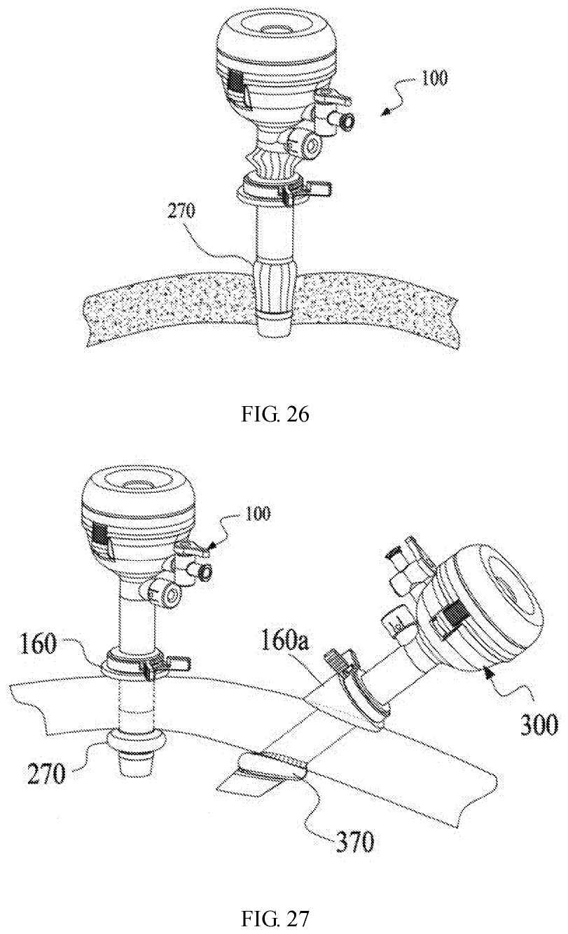

Referring to FIG. 24-26, the balloon body 270 of the balloon assembly 250 is evacuated or folded before the cannula 100 is assembled and packed into the final sterilization package. The slip tube 250 is then moved from the distal end from the proximal end until the balloon body 270 is fully stretched, from the free state to the straight state, and then secured by the external fixation assembly 160. FIG. 26 is a simulation view of the cannula puncturing the body wall of the patient. It will be understood by those skilled in the art that since the balloon body 270 of the cannula 100 is stretched to the straight state, the size change caused by the accumulation of the material of the balloon body 270 is very small, thereby further reducing the puncture resistance. After the cannula 100 completely penetrates the body wall of the patient, the slip tube 250 is moved from the proximal end to the distal end to cause the balloon body 270 to contract and fold back to the free state. The balloon body 270 is then inflated using the syringe SY according to the method described above, and then the external fixation assembly 160 is adjusted so that the balloon body 270 and the gasket 150 respectively clamp the patient's body wall on the inner side and the outer side of the abdominal wall , thereby realizing the fixing of the cannula 100. It is limited as the inflated state. When the operation is completed and the cannula 100 needs to be removed, the one-way plug 145 can be open by the nozzle of the syringe SY and the gas in the balloon body 270 can be evacuated. The slip tube 250 is then moved from the distal end to the proximal end until the balloon body 270 is fully stretched to the straight state, so that the cannula 100 can be easily removed from the body wall.

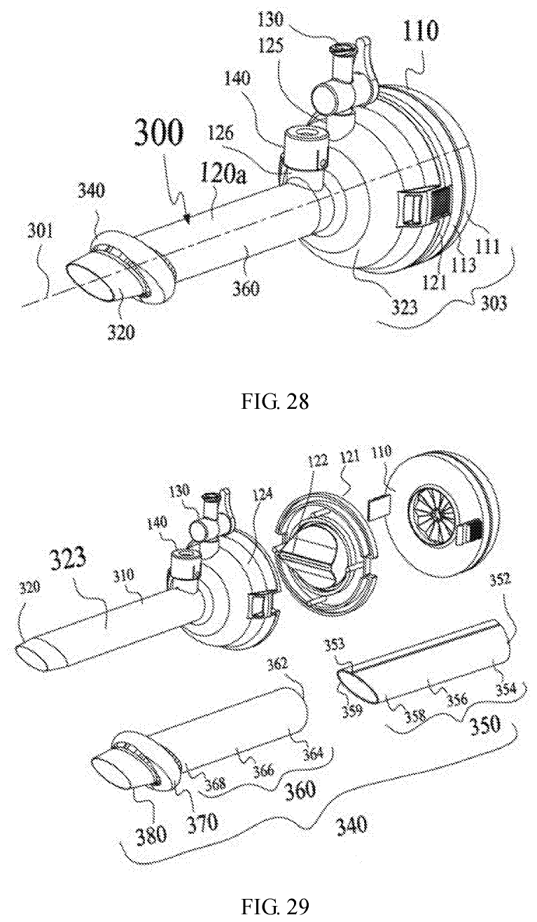

FIG. 28-29 depict the cannula 300 in another embodiment of the present invention. The cannula 300 includes an axis 301 and the first seal assembly 110 and the second seal assembly 120a that are axially placed. The second seal assembly includes the duck bill 122 sandwiched between the lower cover 121 and the lower chamber 323. The lower chamber 323 also includes a lower outer casing 124 and a hollow sleeve 310 coupled thereto and extending distally. The lower chamber 323 also includes a balloon assembly 350, a valve assembly 130 and a one-way valve assembly 340. The gas valve assembly 130 is mounted on the valve mounting hole.

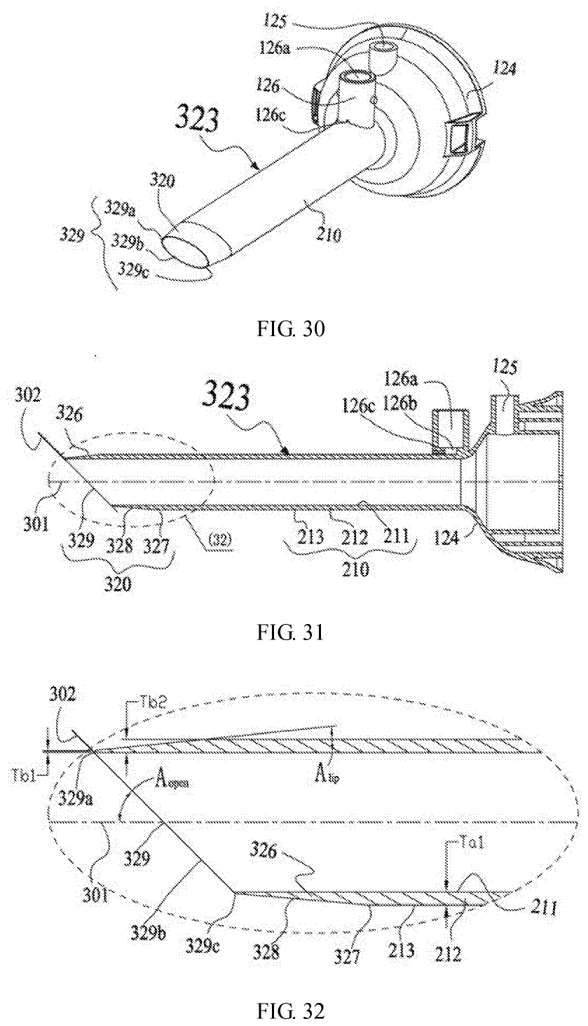

Referring to FIGS. 30-32, the hollow sleeve 310 includes an inner cylinder surface 211 and an outer cylinder surface 213 and a sleeve wall 212 with the thickness Ta1 therebetween; the distal end of the hollow sleeve 310 also includes an open sleeve lip 320 including a slanted cylinder surface 327 between the opening lip 329 and the transitional lip 328, the inner cylinder surface 211 and the slanted cylinder surface 327 limiting the sleeve slanted-wall 326, which has the thickness Tb1 at the opening lip 329 and the thickness Tb2 at the transition lip 328. The thickness of the slanted wall portion 326 gradually increases from the distal end to the proximal end, and generally the increase rate of the thickness is small, resulting in the smoother transition between the obturator and the cannula. In a preferred solution, Tb2=Ta, and 0.1 mm [symbol] Tb1 [symbol] 0.3 mm, and 0.8 mm [symbol] Tb1 [symbol] 1.1 mm. In a preferred solution, the slanted cylinder surface 327 and the outer cylinder surface 211 forms the included angle Alip, wherein 3° [symbol] Alip [symbol] 15°.

With continued referring to FIGS. 30-32, in the present embodiment, the hollow sleeve 310 has the integral wedge-shaped sleeve lip 320, that is, the opening lip 329 and the transverse plane perpendicular to the sleeve axis 301 forms the acute angle. The integral wedge-shaped sleeve lip 320 helps to reduce the puncture force. More precisely, the opening lip 329 includes the distal opening lip 329a and the proximal opening lip 329c and the opening lip connecting segment 329b therebetween. The opening lip 329 limits a mouth plane 302 that includes all or most of the opening lip, the mouth plane 302 intersecting the sleeve axis 301 to form an included angle Aopen. In the present embodiment, the opening lip 329 is completely contained within the mouth plane 302, that is, the opening lip 329 is a 2-dimensional linear ring; while the opening lip 329 may also be 3-dimensional ring, that is, the opening lip 329 is not completely inside a certain plane. When the opening lip 329 is a 3-dimensional ring, the plane that can simultaneously pass through most of the line segment of the opening lip is taken as the mouth plane 302. Although the opening lip 329 in this embodiment is a closed ring, it may be a non-closed ring with one or more small notches.

In an optional solution, the included angle Aopen is an acute angle and 45° [symbol] Aopen [symbol] 85°. Generally, the Aopen angle values are different for different sizes of cannulas, such as Aopen=85° in the 5-mm cannula, Aopen=60° in the 10-mm cannula, and Aopen=45° in the 12-mm cannula. Generally, the larger the Aopen angle value, the better the puncture force is reduced, but the larger the Aopen angle value, the larger the overall length of the cannula and the obturator that needs to be inserted into the patient during puncturing. When 45° [symbol] Aopen [symbol] 85°, different diameters of the cannula can be inserted into the patient in a reasonable depth and with the puncture force as small as possible in a dilemma selected the relatively better balanced solution.

Referring to FIGS. 29 and 33-34, the balloon assembly 340 includes the flexible outer sleeve 360 and the balloon bag lip 380, the balloon body 370 therebetween and the rigid slip tube 350. The balloon body 370 includes the balloon proximal transition region 372, the balloon distal transition region 376 and the balloon body 374 extending therebetween. The balloon lip 380 includes the balloon opening lip 389, the balloon transition lip 387 and the balloon slanted-wall 388 extending therebetween, the balloon slanted-wall 388 limiting the conical bore 386 that matches the shape and size of the sleeve slanted-wall 326. The balloon transition lip 387 extends proximally from the distal end and is seamlessly coupled and smoothly transitioned to the balloon distal transition region 376. The outer sleeve 360 includes the outer-shaft distal-end 368, the outer-shaft proximal-end 364 and the outer-shaft wall 366 extending therebetween, the outer-shaft distal-end 368 being seamlessly coupled and smoothly transitioned to the balloon proximal transition region 372. The outer-shaft wall 366 limits a cylindrical bore 365 that conforms to the shape and size of the outer cylinder surface 213, the outer-shaft proximal-end 364 limiting an outer-shaft opening 362. The slip tube 350 includes the slip-tube distal-end 358, the slip-tube proximal-end 354 and the slip-tube wall 356 extending therebetween. The slip-tube distal-end 358 limits a slip-tube distal opening 359, and the slip-tube proximal end 364 limits the slip-tube proximal opening 352. The slip tube 350 further includes a cutting groove 353 extending therethrough, the cutting groove 353 cutting the slip-tube wall 356 into the open tube-shape with the non-closed circular cross-section. The outer sleeve 360 has an axial length that is greater than the slip tube.

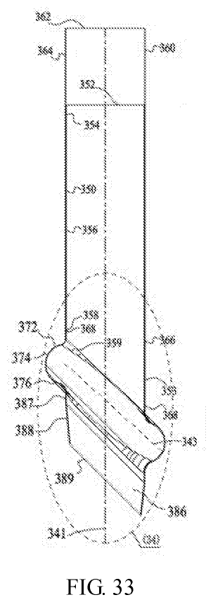

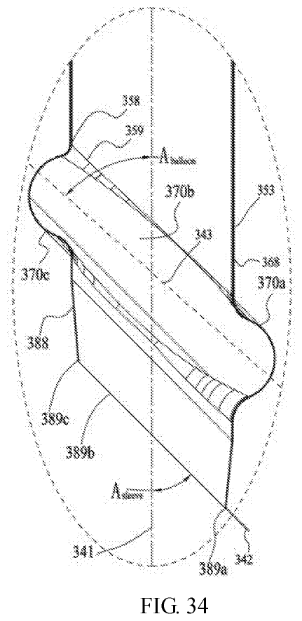

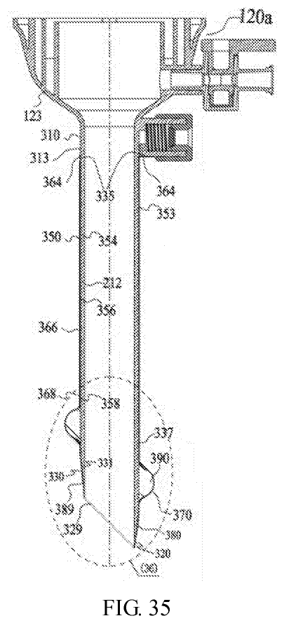

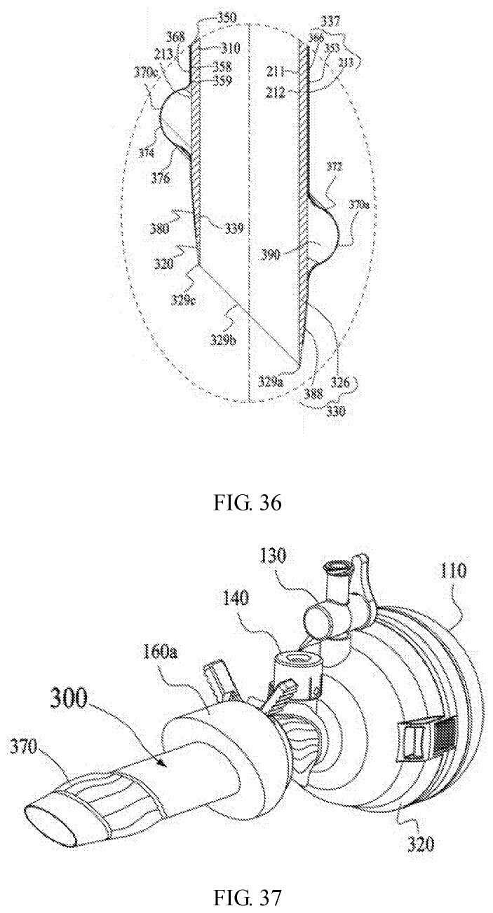

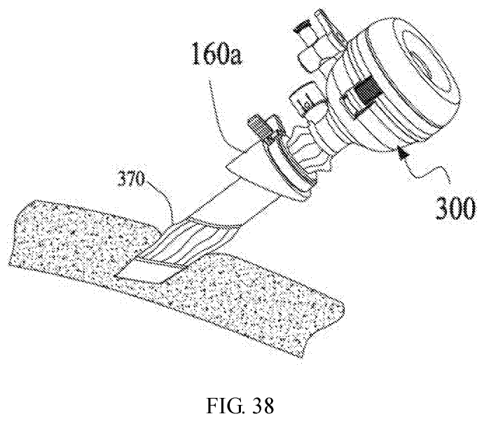

Referring to FIGS. 33-34, the slipping outer tube 350 is mounted inside the outer sleeve 360, wherein the slip-tube distal end 358 is substantially aligned with the outer-shaft distal-end 368, and the outer-shaft proximal-end 364 extends beyond the slip-tube proximal-end 354 continues to extend to the proximal end. Preferably, the slip-tub wall 356 and the outer-shaft wall 366 are all or most fixed together by glue bonding to form the balloon assembly 340. Referring to FIGS. 35-36, the balloon assembly 340 is mounted on the exterior of the hollow sleeve 310, wherein the balloon lip 380 is wrapped around the exterior surface of the sleeve lip 320; all of the slip tube 350 and the proximal end portion of the outer sleeve 360 are wrapped around the exterior of the outer cylinder surface 213. The balloon slanted-wall 388 mates with the sleeve slanted-wall 326 to form a taper fit 330. In one solution, the annular fully closed taper seam region 339 is formed between the balloon slanted-wall 388 and the sleeve slanted-wall 326 by welding (or glue bonding), thereby connecting the balloon lip 380 and the sleeve lip 320 into a whole. The outer-shaft proximal end 364 is bonded to the outer cylinder surface 213 and the exterior surface of the valve seat 326 to form a fully closed proximal seam region 335. The outer cylinder surface 213, the cutting groove 353, and the outer-shaft wall 366 constitute the passageway 337 that communicates with the one-way valve assembly 140 and the balloon body 370. The one-way valve assembly 140, the passageway 337, the outer cylinder surface 213, the balloon assembly 340, the proximal seam region 335 and the taper seam region 339 form a closed balloon cavity 390.

With continued reference to FIGS. 33-36, in the present embodiment, the balloon assembly 250 comprises the integral wedge-shaped balloon lip 280, that is, the balloon opening lip 289 and the transverse plane that is perpendicular to the axis 251 forms the acute angle. More precisely, the opening lip 489 includes the distal balloon opening lip 489a, the proximal balloon opening lip 489c and the balloon connection section 489b therebetween. The opening lip 489 limits a mouth plane 342 that includes all or most of the opening lip, the mouth plane 342 intersecting the sleeve axis 341 to form an included angle Asleeve. In one design, the Asleeve is an acute angle and Asleeve=Aopen, but may not be equal. The balloon body 370 has the ring cavity shaped like the lifebuoy, the ring cavity with the annular-cake shape shaped like including the central hole. In the cannula comprising the balloon that has been disclosed and commercialized so far, the ring cavity (annular-cake) formed by the balloon is substantially perpendicular to the axis of its cannula. While the balloon body 370 in the present invention includes the slanted balloon, that is, the angle formed by the ring cavity (annular-cake) by the balloon body 370 and the axis of the sleeve is an acute angle (not perpendicular). More precisely, the slanted balloon body 370 includes the balloon distal portion 370a and the balloon proximal portion 370c and the balloon middle segment 370b extending therebetween. After the balloon body 370 is inflated, the ring cavity similar to the lifebuoy is formed, making the balloon plane 343 substantially parallel to the balloon body 370, the balloon plane 343 intersecting the axis 341 to form an balloon dip angle Aballoon. In an optional solution, the balloon dip angle Aballoon is an acute angle and 45° [symbol] Aballoon [symbol] 85°. Those skilled will understand in the art that in the direct puncture technique, when the surgeon uses the obturator and the cannula to puncture the abdominal wall of the patient and establish the puncture channel, the axis of the cannula generally obliquely penetrates the abdominal wall (not perpendicular). One of the important functions of obliquely penetrating the abdominal wall is to cover the muscles or tissues at the wounds after the cannula is pulled out , which is beneficial to prevent the incisional hernia complication. The acute angle limits the angle between the cannula axis and the patient's abdominal wall is the puncturing angle (3. Generally, the larger the diameter of the cannula, the smaller the puncture angle selected at the time of puncture, but usually the puncture angle is usually not less than 45°, preferably 45° [symbol] Aballoon [symbol] 85°. When the ring cavity (annular-cake) formed by inflation of the balloon body 270 is substantially parallel to the abdominal wall of the patient at the puncture position, the balloon body 270 has a better fixed effect, so the balloon dig angle is preferably 45° [symbol] Aballoon [symbol] 85.

Referring to FIG. 37, the cannula 300 further includes an external fixation assembly 160a. The structure and function of the external fixation assembly 160 and the external fixation assembly 160 are substantially equivalent, the only difference is that the distal end of the gasket of the assembly 160a includes the dip angle Aclamp,in one design, the Aclamp=Aopen. Referring to FIG. 27, 37 and 38, the functions and usage methods of the cannula 300 are substantially equivalent to the cannula 100, and no more details herein. The balloon assembly 300 comprises the integral wedge-shaped balloon lip 280 and the slanted balloon, which can further reduce the puncture force.

Many different embodiments and examples of the invention have been shown and described. One of those ordinary skilled in the art will be able to make adaptations to the methods and apparatus by appropriate modifications without departing from the scope of the invention. For example, the balloon opening lip 289 and the opening lip 229 can be not aligned. For example, the outer sleeve 260 and the balloon body 270 are cut at the distal end 268 into the first portion (the balloon portion) and the second portion (the appearance portion).The first portion is manufactured by the aforementioned Vacuum pre-shaped blow molding, and the second portion is manufactured by extrusion molding, and the first portion and the second portion are bonded together to form a whole. For example, the cutting groove 259 does not have to completely cut the slip tube 250, but simply cuts the slip-tube proximal-end 254 to be easily deformed. For example, the slip tube 250 may be frictionally fixed or elastically fixed with the outer cylinder surface to generate the certain holding force, and the slip tube may be moved by the hand, while the thrust generated by the compressed gas in the balloon is not enough to move the slip tube. For example, the outer tube sleeve of the slip tube 250 and the balloon body extending along the proximal end is installed inside and outside, that is, the slip tube 250 is completely wrapped in the outer sleeve of the balloon body. It is also can be used that the outer sleeve of the balloon body is cut into two segments, and the slip tube 250 is installed in the middle. Thereby, the connection is combined in the order of left, center, and right, forming the structure in which the inner side includes the gas passage and the balloon body can be stretched by moving the slip tube 250. Several modifications have been mentioned, to those skilled in the art, other modifications are also conceivable. Therefore, the scope of the invention should follow the additional claims, and at the same time, it should not be understood that it is limited by the specification of the structure, material or behavior illustrated and documented in the description and drawings.

What is claimed is:

1. a cannula comprising the movable and inflatable balloon, includes the first seal assembly and the second seal assembly, which comprises the lower outer casing and the hollow sleeve connected thereto and extending to the distal end. The first seal assembly, the second seal assembly and the hollow sleeve include an instrument access that is communicated and substantially aligned, wherein:
1) the hollow sleeve including an inner cylinder surface, an outer cylinder surface and a sleeve-wall therebetween;
2) the cannula further includes an inflatable balloon assembly, an one-way valve assembly for inflation and deflation, and a gas access connecting the balloon assembly and the one-way valve assembly; the cannula is mounted on an outer portion of the hollow sleeve; the balloon lip is wrapped on the exterior surface of the sleeve lip;
3) the balloon assembly further includes a movable mechanism which are moved from the distal end to the proximal end to cause the airbag body stretched from the free state to the straight state when the cannula is inserted or removed; when the puncture is completed, operating the movable mechanism from the proximal end to the distal end to return the balloon body from the straight state to the free state, and then the gas is injected through the gas access to inflate the balloon body.

2. The cannula of claim 1, wherein the movable mechanism includes the outer sleeve extending proximally from the balloon body, and the slip tube wrapped by the outer sleeve; the outer sleeve and the slip tube are aligned at the distal end, the outer sleeve with the axial length Lo, the slip tube with the axial length Li, and Lo>Li.

3. The cannula of claim 1, wherein the sleeve lip including a slanted cylinder surface between the opening lip and the transitional lip, the inner cylinder surface and the slanted cylinder surface limiting the sleeve slanted-wall; the balloon lip extending distally forms the balloon slanted-wall that matches the shape and size of the sleeve slanted-wall; the balloon lip extends proximally to the balloon body connected and smoothly transitioned thereof; the balloon slanted-wall and the sleeve slanted-wall form a taper fit; and the balloon slanted-wall and the sleeve slanted-wall are fixed to form an annular or curved complete-closed taper seam region to connect the balloon lip and the sleeve lip into a whole.

4. The cannula of claim 1, wherein both the balloon lip and the balloon body have the uniform thickness Ta3, and 0.05 mm [symbol] Ta3 [symbol] 0.1 mm.

5. The cannula of claim 1, wherein the balloon body comprises the balloon proximal transition region, the balloon distal transition region, and the balloon body extending therebetween. The balloon lip is seamlessly coupled and smoothly transitioned to the balloon distal transition region. The outer sleeve comprises the outer-shaft distal-end and the outer-shaft proximal-end and the outer-shaft wall extending therebetween. The outer-shaft distal-end is connected to the balloon proximal transition region as a whole.

6. The cannula of claim 1, wherein the balloon plane of the balloon body and the balloon axis forms the included angle Aballoon, and 45° [symbol] Aballoon [symbol] 85°.

7. The cannula of claim 6, wherein the hollow sleeve includes the integral wedge-shaped opening lip, the opening lip limiting the mouth plane, and the angle Aopen which is formed by the mouth plane intersects the axis, and 45° [symbol] Aopen [symbol] 85°.

8. The cannula of claim 7, wherein the cannula further includes an external fixation assembly that includes a gasket and a lock member disposed along the axis. The gasket includes the gasket distal-end thereof, the acute angle Aclamp which is formed by the gasket distal-end intersects the axis, and Aclamp=Aopen.

9. The cannula of claim 5, wherein the outer-shaft proximal-end is fixed to the exterior surface of the outer cylinder surface and the valve seat of the one-way valve assembly to form a fully closed proximal seam region. The gas access connecting the cannula and the one-way valve assembly is formed between the outer sleeve and the outer cylinder surface.

10. The cannula of claim 1, wherein the slanted cylinder surface and the outer cylinder surface forms the included angle Alip, wherein 3° [symbol] Alip [symbol] 15°.
